(19) United States
(12) Patent Application Publication
(10) Pub. No.: US 2016/0150970 A1
(43) Pub. Date: Jun. 2, 2016

(54) PHOTOACOUSTIC APPARATUS

(71) Applicant: CANON KABUSHIKI KAISHA, Tokyo (JP)

(72) Inventors: Minoru Ohkoba, Utsunomiya-shi (JP); Kazuhito Oka, Tokyo (JP)

(21) Appl. No.: 14/948,091

(22) Filed: Nov. 20, 2015

(30) Foreign Application Priority Data
Nov. 28, 2014 (JP) 2014-242452

(51) Int. Cl. A61B 5/00

(52) U.S. Cl. CPC A61B 5/0095; A61B 5/7475; A61B 2576/00

(57) ABSTRACT

A photoacoustic apparatus according to the present invention includes an electrical stimulation unit configured to apply a voltage to a subject, and a processing unit configured to set a light irradiation timing such that the subject is irradiated with pulsed light at least once in a period from an irradiation start timing determined in accordance with an application start timing of the voltage to when a first time period starting from the application start timing and ending at an application end timing of the voltage passes, and to set the light irradiation timing such that the subject is not irradiated with pulsed light outside the period from the irradiation start timing to when the first time period passes.

### BACKGROUND OF THE INVENTION

1. Field of the Invention

The present invention relates to a photoacoustic apparatus using a photoacoustic effect.

2. Description of the Related Art

One of imaging technologies using light is a photoacoustic imaging technology. A subject is first irradiated with pulsed light generated from a light source in photoacoustic imaging. The irradiation light propagates and is diffused through the subject and is absorbed at a plurality of locations inside the subject, thereby generating a photoacoustic wave. A transducer converts this photoacoustic wave into an electrical signal, and a processing apparatus acquires information on optical property values of the inside of the subject by performing analysis processing on the electrical signal.

A generated sound pressure P₀ (hereinafter also referred to as an initial sound pressure) of a photoacoustic wave generated from a light absorber inside the subject may be expressed by the following expression.

P₀=Γ·μₐ·Φ (1)

Here, Γ denotes the Grüneisen coefficient, which is obtained by dividing the product of the volumetric expansion coefficient β and the square of the speed of sound c by the constant pressure specific heat Cₚ. Φ denotes light intensity (the intensity of light that has reached the light absorber, and that may also be referred to as light fluence) at a certain location (a local area).

The initial sound pressure P₀ may be obtained by using a received signal (PA signal) output from a search unit that has received the photoacoustic wave.

When a type of tissue is set, it is known that the Grüneisen coefficient for the tissue has a nearly constant value. Thus, the product of the optical absorption coefficient μₐ and the light intensity Φ, namely, an optical energy absorption density may be obtained by measuring and analyzing, for a plurality of locations, a temporal change in a PA signal at each location.

Japanese Patent Laid-Open No. 2013-248077 discloses a photoacoustic image generation apparatus that generates a photoacoustic image of a blood vessel in accordance with a photoacoustic wave generated because of light.

In a case where it is assumed that hemoglobin is a light absorber, which is an object under measurement, since the amount of hemoglobin is small in an area where the amount of blood in a blood vessel is small, the optical absorption coefficient of the area is relatively low. Thus, the sound pressure of a photoacoustic wave generated in accordance with Expression (1) is low. That is, the signal-to-noise (S/N) ratio of a received signal of a photoacoustic wave generated at the area where the amount of blood is small is relatively low. Thus, when subject information for a target area is acquired by a photoacoustic apparatus, the accuracy of subject information may be low at an area where the amount of blood is small.

### SUMMARY OF THE INVENTION

The present invention provides a photoacoustic apparatus including a light irradiation unit configured to irradiate a subject with pulsed light, a receiving unit configured to receive a photoacoustic wave generated by irradiation of the subject with pulsed light emitted from the light irradiation unit, and output a signal, an electrical stimulation unit configured to apply a voltage to the subject, and a processing unit configured to set a light irradiation timing at which the light irradiation unit irradiates the subject with pulsed light, and acquire subject information in accordance with the signal. The processing unit sets the light irradiation timing such that the subject is irradiated with pulsed light at least once in a period from an irradiation start timing determined in accordance with an application start timing of the voltage to when a first time period starting from the application start timing and ending at an application end timing of the voltage passes, and sets the light irradiation timing such that the subject is not irradiated with pulsed light outside the period from the irradiation start timing to when the first time period passes.

Further features of the present invention will become apparent from the following description of exemplary embodiments with reference to the attached drawings.

### BRIEF DESCRIPTION OF THE DRAWINGS

FIG. 1 is a diagram of the configuration of a photoacoustic apparatus according to a first embodiment.

FIG. 2 is a diagram illustrating a flowchart of a subject-information acquisition method according to the first embodiment.

FIG. 3 includes diagrams illustrating various types of sequence according to the first embodiment.

FIG. 4 includes other diagrams illustrating various types of sequence according to the first embodiment.

FIG. 5 includes other diagrams illustrating various types of sequence according to the first embodiment.

FIG. 6 illustrates a flowchart of a subject-information acquisition method according to a second embodiment.

FIG. 7 illustrates a flowchart of a subject-information acquisition method according to a third embodiment.

### DESCRIPTION OF THE EMBODIMENTS

In the following, the present invention will be described in greater detail with reference to the drawings. Note that, as a general rule, the same constructional elements are denoted by the same reference numerals, and the description thereof will be omitted.

### First Embodiment

A photoacoustic apparatus according to the present embodiment is an apparatus that acquires subject information in accordance with received signals of photoacoustic waves. The subject information according to the present embodiment is information on a subject obtained from received signals of photoacoustic waves generated by a photoacoustic effect. Specifically, the subject information includes, for example, a generated sound pressure (initial sound pressure), an optical energy absorption density, an optical absorption coefficient, and concentrations of substances that constitute certain tissue. Here, the concentrations of substances include, for example, oxygen saturation, oxyhemoglobin concentration, deoxyhemoglobin concentration, and total hemoglobin concentration. The total hemoglobin concentration is the sum of the oxyhemoglobin concentration and the deoxyhemoglobin concentration. In addition, distribution data such as an optical-absorption-coefficient distribution and an oxygen-saturation distribution may be treated as subject information.

### (Basic Configuration)

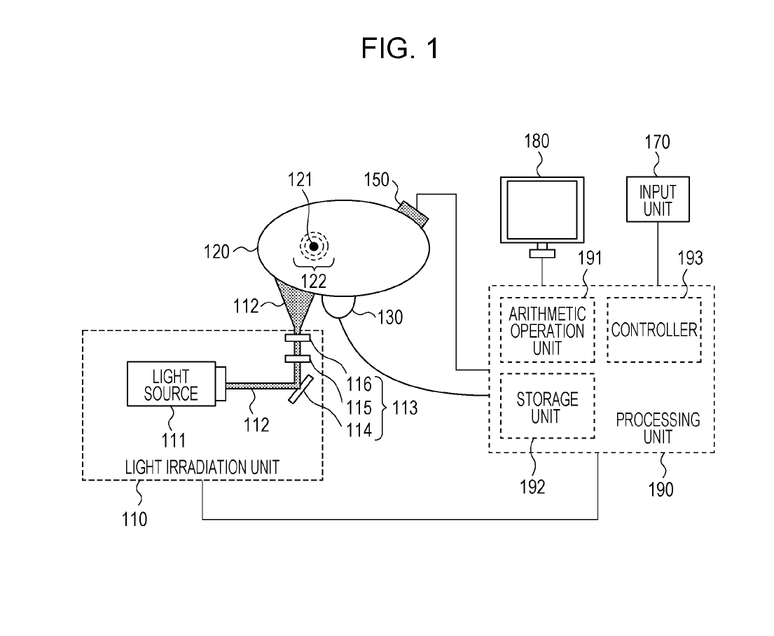

A basic configuration of the photoacoustic apparatus according to the present embodiment will be described with reference to FIG. 1.

FIG. 1 is a schematic diagram illustrating the configuration of the photoacoustic apparatus according to the present embodiment. The photoacoustic apparatus according to the present embodiment includes a light irradiation unit 110, an acoustic-wave receiving unit 130, an electrical stimulation unit 150, an input unit 170, a display unit 180, and a processing unit 190. The light irradiation unit 110 includes a light source 111 and an optical system 113. Note that the configurations of these elements will be described later in detail.

First, pulsed light 112 emitted from the light source 111 is guided by the optical system 113. A subject 120 is irradiated with the pulsed light 112 emitted from the optical system 113, and the pulsed light 112 reaches a light absorber 121 inside the subject 120. As the light absorber 121, blood vessels in the living body are typically used, and in particular, for example, hemoglobin existing in the blood vessels is used. The light absorber 121 absorbs the energy of light and generates photoacoustic waves 122. The generated photoacoustic waves 122 propagate through the subject 120, and reach the acoustic-wave receiving unit 130.

The acoustic-wave receiving unit 130 outputs time-series received signals by receiving the photoacoustic waves 122. Received signals output from the acoustic-wave receiving unit 130 are sequentially input to the processing unit 190.

The processing unit 190 generates subject information for a target area, using the input time-series received signal. Then, the processing unit 190 transmits data of the generated subject information to the display unit 180, and causes the display unit 180 to display an image or numerical values based on the subject information for the target area. Note that the target area may be preset, or may also be input by a user using the input unit 170. The target area is set so as to include at least part of the subject 120. Note that a subject-information acquisition method will be described in detail later.

When it is assumed that hemoglobin is a light absorber, since the amount of hemoglobin is small in an area where the amount of blood in a blood vessel is small, the optical absorption coefficient of the area is relatively low. Thus, the sound pressure of a photoacoustic wave generated in accordance with Expression (1) is low. That is, the signal-to-noise (S/N) ratio of a received signal of a photoacoustic wave generated at the area where the amount of blood is small is relatively low. In addition, in the case where the amount of blood is significantly small, a received signal of a photoacoustic wave may be lost in noise. Thus, when subject information for a target area is acquired by a photoacoustic apparatus, the accuracy of subject information may be low at an area where the amount of blood is small.

It is known that blood staying in a blood vessel is pumped by a muscle pump (muscle contraction), which is for example the calf muscle, and the blood flows. However, in a living body where the effect of pumping performed by a certain muscle pump is small and the intensity of muscle contractions is low, it is considered that the amount of pumped blood is small and thus blood vessels having a small amount of blood may exist. In addition, since the amount of blood pumped by the muscle pump such as the calf muscle is not constant, the amount of blood changes in time sequence.

In light of the above-described problems, the photoacoustic apparatus according to the present embodiment includes the electrical stimulation unit 150, which gives electrical stimulation. The electrical stimulation unit 150 may cause a stimulation-target region to compulsorily perform pumping by giving electrical stimulation to the stimulation-target region. Then, the amount of blood in veins and peripheral blood vessels may be increased by pumping caused by the electrical stimulation unit 150. That is, it is considered that after the electrical stimulation unit 150 has given electrical stimulation to a certain stimulation-target region, a change in blood flow occurs and the amount of blood for a target area temporarily increases. Thus, the processing unit 190 sets the light irradiation timing of the light irradiation unit 110 such that the subject 120 is irradiated with light when the amount of blood for a target area increases after the electrical stimulation unit 150 has given electrical stimulation to a certain stimulation-target region. That is, the processing unit 190 sets the light irradiation timing of the light irradiation unit 110 such that the subject 120 is not irradiated with light when the amount of blood for the target area is small after the electrical stimulation unit 150 has given electrical stimulation to the certain stimulation-target region.

In this manner, light irradiation may be selectively performed when the amount of blood is large, that is, the amount of hemoglobin serving as a light absorber is large, and thus many received signals of photoacoustic waves having high S/N ratios may be acquired. Then, according to the present embodiment, there is a low probability that received signals of photoacoustic waves generated when the amount of blood is small and having low S/N ratios are contained. These make it possible to acquire subject information for a target area with high accuracy in accordance with received signals having high S/N ratios. Note that details of the light irradiation timing will be described later.

In the following, constitutional blocks of the photoacoustic apparatus according to the present embodiment will be described.

### (Light Source 111)

The light source 111 can be a pulsed light source capable of generating pulsed light having the pulse width on the order of nanoseconds to microseconds. Specifically, a pulse width of about 1 to 100 nanoseconds can be used. In addition, wavelengths in the range from 400 nm to about 1600 nm can be used. In particular, light having a visible-light wavelength (greater than or equal to 400 nm but not greater than 700 nm) can be used when high-resolution imaging of a blood vessel near the surface of the living body is performed. In contrast, when imaging of a portion deep inside the living body is performed, light having a wavelength that is not absorbed much by the background tissue of the living body (greater than or equal to 700 nm but not greater than 1100 nm) can be used. Note that the ranges of terahertz waves, microwaves, and radio waves may also be used.

Specifically, a laser can be used as the light source 111. In addition, when measurement is performed using light having a plurality of wavelengths, lasers capable of changing wavelengths to be oscillated can be used. Note that in the case where the subject 120 is irradiated with light having a plurality of wavelengths, a plurality of lasers oscillating respective wavelengths that differ from each other may be used by performing switching of oscillation for each of the lasers or by alternately performing irradiation. In the case where a plurality of lasers are used, too, the lasers are altogether expressed as a light source.

Various lasers such as solid-state lasers, gas lasers, dye lasers, and semiconductor lasers may be used as the lasers. In particular, pulsed lasers such as Nd:YAG lasers and alexandrite lasers can be used. In addition, Ti:sa lasers using Nd:YAG laser light as excitation light and optical parametric oscillator (OPO) lasers may also be used. In addition, for example, light-emitting diodes may be used instead of the lasers.

### (Optical System 113)

The optical system 113 causes the pulsed light 112 to travel from the light source 111 to the subject 120. Optical elements such as lenses, mirrors, and optical fibers may be used as the optical system 113. In addition, the optical system 113 according to the present embodiment includes an optical mirror 114 for changing the direction of travel of the pulsed light 112, a light adjustment unit 115, and a diffuser 116.

A light emission unit of the optical system 113 can increase the beam diameter of the pulsed light 112 using, for example, the diffuser 116 and perform irradiation in a living-body information acquisition apparatus treating, for example, the breast as a subject. In contrast, the light emission unit of the optical system 113 can be constituted by lenses and the like, and can perform irradiation by focusing the beam diameter to increase resolution in a photoacoustic microscope.

In addition, the optical system 113 may include the light adjustment unit 115, which is capable of adjusting the amount of attenuation of the pulsed light 112 emitted from the light source 111. Every unit capable of adjusting the amount of attenuation of the pulsed light 112 such as a mechanical shutter or a liquid crystal shutter may be used as the light adjustment unit 115. Note that, herein, “pulsed-light irradiation is not performed” indicates not only the case where the intensity of pulsed light with which irradiation is to be performed is set to zero but also the case where pulsed-light irradiation is performed whose intensity has been attenuated to a level at which it is difficult to detect a photoacoustic wave generated by the pulsed light. It can be said that, in the present invention, the optical system 113, which adjusts the amount of attenuation of the pulsed light 112, performs weighting on the amount of attenuation of the pulsed light 112 in accordance with the amount of blood estimated from an electrocardiogram signal.

In addition, the optical system 113 may be moved with respect to the subject 120, which makes it possible to image a wide range of the subject 120.

Note that the subject 120 may be irradiated with light directly by the light source 111 without using the optical system 113.

### (Subject 120)

Although the subject 120 is not part of the photoacoustic apparatus according to the present invention, the subject 120 will be described in the following. The main purpose of the photoacoustic apparatus according to the present embodiment is, for example, to examine a malignant tumor or a blood-vessel disease of a person or animal, and follow-up after chemotherapy treatments. Thus, a living body, or specifically, regions such as the breast, the neck, or the abdomen of a human body or an animal may be the subject 120.

In addition, a substance having a relatively high optical absorption coefficient inside the subject 120 can be used as the light absorber 121 inside the subject 120. For example, when a human body is a measurement target, a blood vessel containing a lot of oxyhemoglobin, deoxyhemoglobin, or both or a newborn blood vessel formed near a tumor is the light absorber 121.

### (Acoustic-Wave Receiving Unit 130)

The acoustic-wave receiving unit 130 includes one or more conversion elements and a housing. As each of the conversion elements, any conversion element may be used as long as the conversion element is capable of receiving an acoustic wave and converting the acoustic wave into an electrical signal. Examples of such a conversion element are a piezoelectric element using a piezoelectric phenomenon of, for example, lead zirconate titanate (PZT), a conversion element using optical resonance, and a capacitance conversion element such as a capacitive micromachined ultrasonic transducer (CMUT). In the case where a plurality of conversion elements are included, the plurality of conversion elements can be arranged as in an arrangement called a 1D array, a 1.5D array, a 1.75D array, or a 2D array, so as to align in a flat surface or a curved surface.

In addition, the acoustic-wave receiving unit 130 can be configured to be moved by a scanning mechanism (not illustrated) with respect to the subject 120 in order to acquire a wide range of subject information. In addition, the optical system 113 (an irradiation position of the pulsed light 112) and the acoustic-wave receiving unit 130 can be moved by synchronizing with each other.

In addition, in the case where the acoustic-wave receiving unit 130 is a hand-held acoustic-wave receiving unit, the acoustic-wave receiving unit 130 has a grip portion for a user to grip the acoustic-wave receiving unit 130. In addition, a receiving surface of the acoustic-wave receiving unit 130 may be provided with an acoustic lens. In addition, the acoustic-wave receiving unit 130 may be provided with a plurality of conversion elements.

In addition, the acoustic-wave receiving unit 130 may be provided with an amplifier that amplifies time-series analog signals output from the conversion elements.

### (Electrical Stimulation Unit 150)

The electrical stimulation unit 150 is a device that gives electrical stimulation to a stimulation-target region and causes the stimulation-target region to perform pumping. Typically, the electrical stimulation unit 150 includes a stimulation-use electrode and a power source used for application of voltage to the stimulation-use electrode. The voltage is applied from the power source to the stimulation-use electrode, and electrical stimulation is given from the stimulation-use electrode to a stimulation-target region. For example, an electrical stimulation system as described in Japanese Patent Laid-Open No. 2014-133123 may be used as the electrical stimulation unit 150.

The electrical stimulation unit 150 may give single non-periodic electrical stimulation, and also give periodic electrical stimulation.

### (Input Unit 170)

The input unit 170 receives various types of input from the user (mainly an examiner such as a health-care worker), and transmits input information to units such as the processing unit 190 via a system bus. For example, through the input unit 170, the user may set parameter settings regarding imaging, command start of imaging, set observation parameter settings such as the range and shape of a target area, and perform other image processing operations regarding images.

The input unit 170 may comprise a mouse, a keyboard, a touch panel, or the like, and notifies a software program such as an operating system (OS) running on a controller 193 of an event in accordance with an operation performed by the user. In addition, the hand-held photoacoustic apparatus can be provided with the input unit 170, which is used to command driving of the light irradiation unit 110. A button-type switch provided at a probe or a foot switch may be used as the input unit 170.

### (Display Unit 180)

A liquid crystal display (LCD), a cathode ray tube (CRT), an organic electroluminescent (EL) display, or the like may be used as the display unit 180. Note that the display unit 180 may not be included in the photoacoustic apparatus according to the present embodiment, and may be prepared separately and connected to the photoacoustic apparatus.

### (Processing Unit 190)

The processing unit 190 serving as a computer includes an arithmetic operation unit 191, a storage unit 192, and the controller 193.

The arithmetic operation unit 191 collects a time-series analog received signal output from the acoustic-wave receiving unit 130, and performs signal processing such as amplification of the analog received signal, analog-to-digital (AD) conversion of the analog received signal, and storing of the digitized received signal. Generally, a circuit called a data acquisition system (DAS) may be used as the arithmetic operation unit 191 performing such processing. Specifically, the arithmetic operation unit 191 includes an amplifier that amplifies a received signal, and an AD converter that digitizes an analog received signal, and the like.

In addition, the arithmetic operation unit 191 may acquire, using received signals, generated-sound-pressure information at positions in the subject 120. The generated-sound-pressure information obtained at positions inside the subject 120 is also called an initial-sound-pressure distribution inside the subject 120. Note that in the case where the photoacoustic apparatus is a photoacoustic tomography apparatus, the arithmetic operation unit 191 may obtain generated sound pressure data corresponding to a position as two-dimensional or three-dimensional space coordinates by reconstructing an image using the acquired received signals. The arithmetic operation unit 191 may use, as an image reconstruction method, a known reconstruction method such as a universal back-projection (UBP) method, a filtered back projection (FBP) method, or a model-based method. In addition, the arithmetic operation unit 191 may also use phasing-and-addition (delay-and-sum) processing as an image reconstruction method.

In addition, after performing envelope detection on the acquired received signals, the arithmetic operation unit 191 may plot, in the directional direction of a certain conversion element (typically in the direction of depth), an amplitude value obtained in a time-axis direction in the resulting signal. The arithmetic operation unit 191 performs this plotting for each of the positions of the conversion elements, and thus may acquire initial-sound-pressure distribution data. In particular, in the case where the photoacoustic apparatus is a photoacoustic microscope, this method can be used.

A processor such as a central processing unit (CPU) or a Graphics Processing Unit (GPU), or an arithmetic-operation circuit such as a field programmable gate array (FPGA) chip may be used as the arithmetic operation unit 191, which performs processing for acquiring generated-sound-pressure information. Note that the arithmetic operation unit 191 may not only be constituted by one processor or one arithmetic-operation circuit but also be constituted by a plurality of processors or a plurality of arithmetic-operation circuits.

The storage unit 192 may store received signals obtained after AD conversion, various types of distribution data, displayed-image data, various types of measurement parameters, and the like. In addition, processes performed using a subject-information acquisition method to be described later may be stored, in the storage unit 192, as programs to be executed by the controller 193 inside the processing unit 190. Note that the storage unit 192, in which the programs are stored, is a non-temporary recording medium. The storage unit 192 is typically constituted by a storage medium, examples of which are a first-in first-out (FIFO) memory, a read-only memory (ROM), a random-access memory (RAM), and a hard disk. Note that the storage unit 192 may not only be constituted by one storage medium but also be constituted by a plurality of storage mediums.

In addition, the processing unit 190 includes the controller 193 for controlling operations of the constitutional blocks of the photoacoustic apparatus. The controller 193 supplies control signals and data necessary for the constitutional blocks of the photoacoustic apparatus via a bus. Specifically, the controller 193 supplies a light-emission control signal for commanding the light source 111 to emit light, a reception control signal for the conversion elements inside the acoustic-wave receiving unit 130, and the like. Typically a CPU is used as the controller 193.

Note that the units included in the processing unit 190 may be constituted as an integral device or may also be constituted as separate devices. In addition, the arithmetic operation unit 191 and the controller 193 may be constituted as a single device. That is, the processing unit 190 may include a single device having the function of the arithmetic operation unit 191 and the function of the controller 193.

### [Subject-Information Acquisition Method]

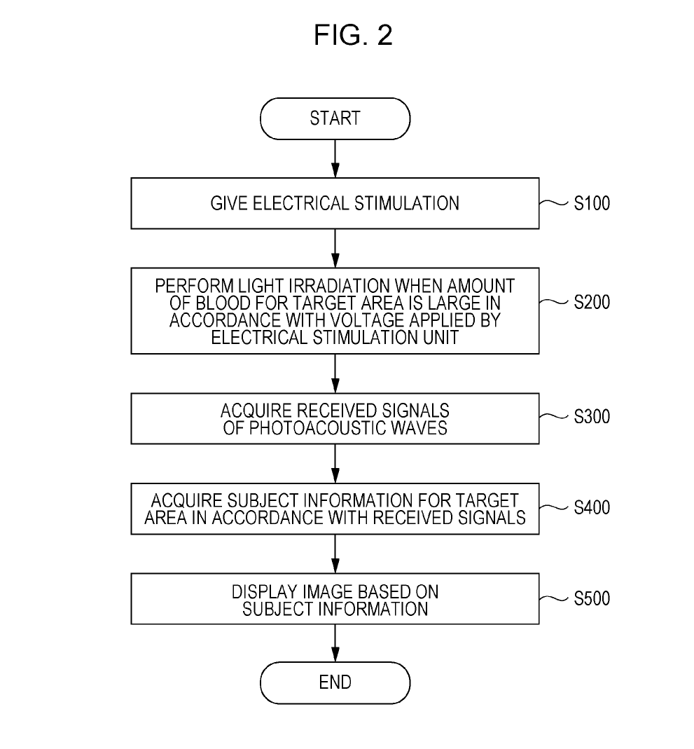

Next, a flowchart for acquiring subject information using the photoacoustic apparatus according to the present embodiment will be described using FIG. 2. The controller 193 reads out a program that is stored in the storage unit 192 and in which the subject-information acquisition method is described, and causes the photoacoustic apparatus to execute the following subject-information acquisition method.

### (S100: Step for Giving Electrical Stimulation)

In this step, the electrical stimulation unit 150 gives electrical stimulation to a stimulation-target region of the subject 120. Here, information on a voltage applied by the electrical stimulation unit 150 is transmitted to the processing unit 190, and is used as a light-irradiation trigger signal in S200. In this step, the stimulation-use electrode of the electrical stimulation unit 150 is positioned as appropriate at a position from which electrical stimulation may be given to the stimulation-target region. For example, in the case where a target area is the right thigh, stimulation-use electrodes of the electrical stimulation unit 150 may be arranged so as to sandwich the right calf.

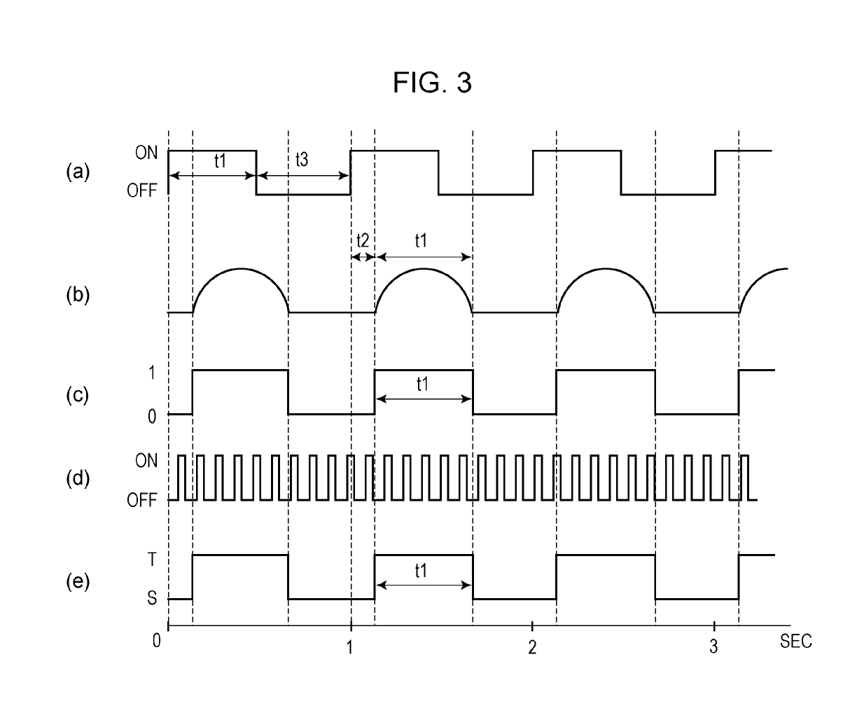

Part (a) of FIG. 3 illustrates an example of a waveform of a voltage applied by the electrical stimulation unit 150. The applied voltage illustrated in Part (a) of FIG. 3 is a periodic applied voltage, the cycle of which is constituted by an application time t1 of 0.5 seconds and a non-application time t3 of 0.5 seconds. The period during which the electrical stimulation unit 150 applies a voltage corresponds to a contractile period of the muscle of the stimulation-target region, and is considered to be a period during which blood is pumped. The period during which the electrical stimulation unit 150 does not apply a voltage corresponds to a relaxation period of the muscle of the stimulation-target region. Note that, herein, a time t1 starting from an application start timing and ending at an application end timing of the voltage applied by the electrical stimulation unit 150 is also called an “application time”. In addition, herein, a time t3 starting from the application end timing and ending at the next application start timing of the voltage applied by the electrical stimulation unit 150 is also called a “non-application time”.

Part (b) of FIG. 3 is a graph illustrating a change in the amount of blood for the target area. For Part (b) of FIG. 3, the case is assumed where a change in the amount of blood caused by factors other than electrical stimulation is small.

As understood from Part (b) of FIG. 3, the amount of blood for the target area does not increase at the application start timing of the voltage but increases after some delay, which is a time t2, from the application start timing of the voltage, the time t2 being required for the blood flow caused by electrical stimulation to reach the target area. It is considered that a period during which the amount of blood is large is maintained for the application time t1, which is a voltage application time, after the time t2 has passed from the application start timing of the voltage.

Herein, the time t2 starting from the application start timing of the voltage and ending at the time when the blood flow caused by electrical stimulation reaches the target area is also called a “delay time”.

(S200: Step for Performing Light Irradiation when Amount of Blood for Target Area is Large in Accordance with Voltage Applied by Electrical Stimulation Unit)

In this step, the controller 193 determines the timing at which the amount of blood for the target area is large because of electrical stimulation, in accordance with the voltage applied by the electrical stimulation unit 150. Then, the controller 193 sets the light irradiation timing of the light irradiation unit 110 such that the light irradiation unit 110 irradiates the subject 120 with the pulsed light 112 at the timing. In contrast, the controller 193 sets the light irradiation timing of the light irradiation unit 110 such that the light irradiation unit 110 does not irradiate the subject 120 with the pulsed light 112 when the amount of blood for the target area is small.

The controller 193 supplies an irradiation control signal to the light irradiation unit 110 in accordance with the voltage applied in S100. The light irradiation unit 110 irradiates the subject 120 with the pulsed light 112 at the light irradiation timing based on the irradiation control signal supplied from the controller 193. The pulsed light 112 is absorbed by the subject 120, and photoacoustic waves 122 are generated by a photoacoustic effect.

Part (c) of FIG. 3 illustrates an example of the irradiation control signal supplied by the controller 193 to the light irradiation unit 110. Irradiation is performed in the state of “1” and is not performed in the state of “0” in Part (c) of FIG. 3. The controller 193 supplies the irradiation control signal such that the light irradiation unit 110 irradiates the subject 120 with the pulsed light 112 in a period from when the delay time t2 passes from the application start timing of the voltage to when the voltage application time t1 passes. That is, the controller 193 supplies the irradiation control signal such that light irradiation is performed in a period during which the blood flow caused by electrical stimulation exists at the target area and the amount of blood is large. In contrast, the controller 193 supplies the irradiation control signal such that light irradiation is not performed outside the period during which the amount of blood is large. Note that the controller 193 may detect the application start timing of the voltage by receiving information on the applied voltage from the electrical stimulation unit 150.

Since the speed of light is extraordinary faster than the speed of photoacoustic waves, it may be considered that photoacoustic waves are simultaneously generated at the timing of irradiation of the pulsed light 112 at certain positions within the target area. Herein, the timing at which the subject 120 is irradiated with the pulsed light 112 is treated as the timing at which photoacoustic waves caused by the pulsed light 112 are generated.

In addition, typically, the voltage application time t1 is set to a time greater than or equal to 10 ms but not greater than 1000 ms. Thus, the light irradiation unit 110 may perform light irradiation in the period from when the delay time t2 passes from the application start timing of the voltage to when a time greater than or equal to 10 ms but not greater than 1000 ms passes, as the period during which the amount of blood is large.

Note that the blood flow caused by electrical stimulation reaches the target area after a time obtained by dividing the length of a blood vessel between the stimulation-target region and the target area by the speed of the blood flow passes from the time when the application start timing of the voltage is detected. Thus, the controller 193 may determine a light irradiation start timing in accordance with the application start timing of the voltage, information on the length of the blood vessel between the stimulation-target region and the target area, and information on the speed of the blood flow. Note that since it is necessary to measure, for each subject, the distance between a stimulation-target region and a target area and the speed of a blood flow in order to determine a light irradiation start timing as described above, the size of the apparatus may be large. Here, the blood flow indicates propagation of pulse waves, which are blood-pressure waves generated because of electrical stimulation.

The light irradiation start timing can be selected from among timings predetermined for respective stimulation-target regions and target-area anatomical regions. That is, the storage unit 192 can have a relationship table regarding the type of stimulation-target region, the type of target-area anatomical region, and the delay time t2. In addition, the photoacoustic apparatus can have the input unit 170 configured to receive the type of stimulation-target region and the type of target-area anatomical region input by the user. For example, the input unit 170 may be configured such that the user is allowed to select the type of stimulation-target region and the type of target-area anatomical region from among a plurality of types of anatomical region displayed on the display unit 180. Then, the controller 193 may read out, from the relationship table stored in the storage unit 192, the delay time t2 corresponding to the types of anatomical region input through the input unit 170. The controller 193 detects the application start timing of the voltage, and may start light irradiation after the delay time t2 read out from the storage unit 192 has passed from the application start timing of the voltage.

Note that, here, the type of stimulation-target region and the type of target-area anatomical region have been described as information necessary to determine the delay time t2; however, the information necessary to determine the delay time t2 is not limited thereto. For example, it is considered that the delay time t2 changes depending on, for example, the age of the subject 120 even when the same type of stimulation-target region and the same type of target-area anatomical region are used. Thus, the input unit 170 can be configured to receive information such as the age of the subject 120 in addition to the type of stimulation-target region and the type of target-area anatomical region. That is, the input unit 170 can be configured to receive at least the type of stimulation-target region and the type of target-area anatomical region. Then, the controller 193 can read out, from the relationship table, the delay time t2 corresponding to received information such as the age of the subject 120.

In addition, in the case where a target anatomical region of the photoacoustic apparatus has been predetermined, the storage unit 192 can store information on the delay time t2 that has been obtained in advance.

Note that in the case where the period from the application start timing of the voltage to when the blood flow caused by electrical stimulation reaches the target area may be ignored, the application start timing of the voltage may be treated as the light irradiation start timing. That is, the delay time t2 may be 0 in this case.

In addition, the light irradiation unit 110 may perform irradiation of only one pulse of light, or may also perform irradiation of a plurality of pulses of light in the period during which the amount of blood is large. That is, the light irradiation unit 110 performs irradiation of at least one pulse of the pulsed light 112 in the period during which the amount of blood is large.

Note that in the case where the light source 111 is a lamp excitation solid-state laser in which heat is likely to be generated, light emission can be performed at a certain repetition frequency to stably drive the light source 111. Then, the light irradiation timing for the subject 120 can be controlled by the optical system 113. Part (d) of FIG. 3 illustrates a driving sequence of the light source 111, and illustrates that light is emitted during “on” and light is not emitted during “off”. As understood from Part (d) of FIG. 3, the light source 111 emits light at a certain repetition frequency (10 Hz) as described above. In addition, Part (e) of FIG. 3 illustrates a driving sequence of the optical system 113, and illustrates that the light adjustment unit 115 let light emitted from the light source 111 pass therethrough at “T” and the light adjustment unit 115 blocks light emitted from the light source 111 at “S”. In this manner, the light irradiation unit 110 may irradiate the subject 120 selectively with the pulsed light 112 in accordance with the irradiation control signal illustrated in Part (c) of FIG. 3.

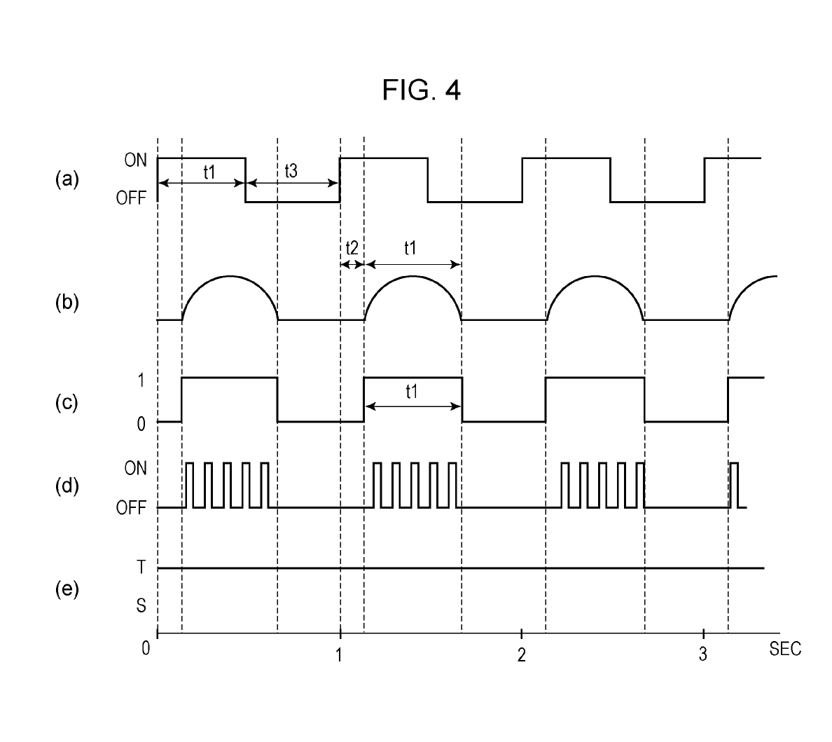

Note that a light-irradiation-timing control method performed by the light irradiation unit 110 is not limited to the method illustrated in Parts (a) to (e) of FIG. 3. As long as light irradiation may be selectively performed when the amount of blood is large, any method may be used. For example, as illustrated in FIG. 4, the light irradiation timing may be controlled by the controller 193 controlling the light-emission timing of the light source 111. The types of sequence illustrated in Parts (a) to (e) of FIG. 4 are the same as those illustrated in Parts (a) to (e) of FIG. 3. As understood from Part (d) of FIG. 4, the light source 111 is controlled by the controller 193 such that the light source 111 does not emit light when the amount of blood is small and emits light when the amount of blood is large. In contrast, the light adjustment unit 115 is controlled by the controller 193 such that the pulsed light 112 generated by the light source 111 always passes through the light adjustment unit 115. Note that, in this case, the light irradiation unit 110 does not have to include the light adjustment unit 115. In addition, in this case, since the number of times of light emission of the light source 111 may be reduced, degradation of the parts constituting the light source 111 may be reduced.

In addition, as in a handheld photoacoustic apparatus, assume a case where the input unit 170 that serves as a switch receiving a command for driving the light irradiation unit 110 from the user is provided. In this case, the light irradiation unit 110 can be configured such that irradiation of the pulsed light 112 is performed only when the amount of blood is large in accordance with an electrocardiogram signal, and information regarding the command for driving the light irradiation unit 110 has been received from the switch. That is, the light irradiation unit 110 can be configured such that light irradiation is not performed when the amount of blood is small, or when information regarding the command for driving the light irradiation unit 110 is not received from the switch. Here, “reception of information regarding the command for driving” includes both acquisition of a signal indicating that the light irradiation unit 110 is to be caused to drive and acquisition of a signal indicating that the light irradiation unit 110 is not to be caused to drive.

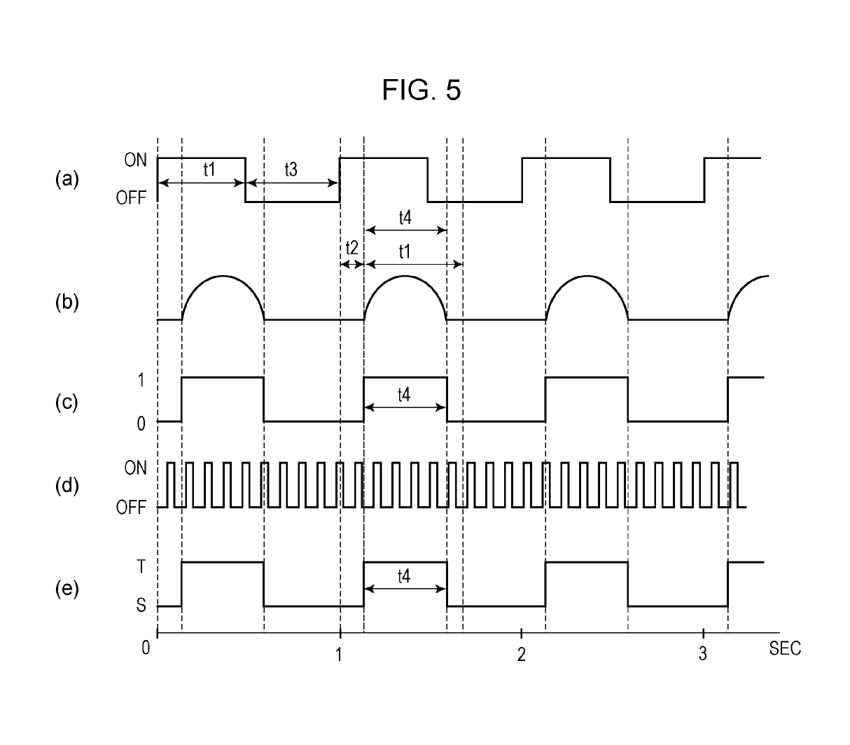

Note that it is considered that the amount of blood is large during the period of the voltage application time t1 after the delay time t2 has passed from the application start timing of the voltage, and the light irradiation timing is set in the present embodiment; however, the setting of the light irradiation timing is not limited to this. For example, the voltage application time t1 corresponds to a contractile period of a certain muscle; however, there may be the case where, depending on the amount of blood staying in a certain blood vessel, most of the blood has been pumped before the voltage application time t1 has passed. That is, there may be the case where a time corresponding to the contractile period of the muscle does not match a time necessary for pumping the blood. In this case, as illustrated in Part (b) of FIG. 5, the amount of blood may increase only during a time t4 shorter than the voltage application time t1. In this case, as illustrated in Part (c) of FIG. 5, the controller 193 can control the light irradiation unit 110 such that irradiation of the pulsed light 112 is performed at least once before the time t4 has passed after the delay time t2 has passed from the application start timing of the voltage. That is, the controller 193 can control the light irradiation unit 110 such that the light irradiation unit 110 performs irradiation of the pulsed light 112 the intensity of which is higher in a period from when the delay time t2 passes from the application start timing of the voltage to when half the voltage application time t1 passes than in a period from when the half the voltage application time t1.

### (S300: Step for Acquiring Received Signals of Photoacoustic Waves)

In this step, the acoustic-wave receiving unit 130 receives the photoacoustic waves 122 generated by the pulsed light 112 with which irradiation is performed in S200, and outputs time-series analog received signals. The arithmetic operation unit 191 collects the time-series analog received signals output from the acoustic-wave receiving unit 130, and performs signal processing such as amplification of the analog received signals and AD conversion of the analog received signals. Then, the arithmetic operation unit 191 stores the digitized received signals in the storage unit 192. The time-series received signal data stored in the storage unit 192 is also called photoacoustic data. A received signal conceptually includes an analog signal and a digital signal in the present invention. In this step, a signal having a high S/N ratio may be selectively acquired, the signal being obtained by performing irradiation of the pulsed light 112 when the amount of blood for the target area is large.

### (S400: Step for Acquiring Subject Information for Target Area in Accordance With Received Signals)

In this step, the arithmetic operation unit 191 serving as a processing unit acquires subject information for the target area in accordance with the received signals acquired in S300. In the present embodiment, the arithmetic operation unit 191 calculates, in accordance with the received signals stored in the storage unit 192, generated-sound-pressure information about photoacoustic waves 122 at certain positions within the target area, namely, an initial-sound-pressure distribution as the subject information, and stores the initial-sound-pressure distribution in the storage unit 192.

Since the initial-sound-pressure distribution obtained in this step is calculated in accordance with the received signals having high S/N ratios and obtained in S300, the accuracy of the initial-sound-pressure distribution is high. Thus, when the arithmetic operation unit 191 causes the display unit 180 to display an image of the initial-sound-pressure distribution stored in the storage unit 192, an image having high image quality such as a high resolution and a high contrast may be presented to the user.

Note that the arithmetic operation unit 191 may also calculate the light fluence of the pulsed light 112 that has reached the positions within the target area, namely, a light-intensity distribution. In the present embodiment, the arithmetic operation unit 191 is capable of acquiring information on the light-intensity distribution of the pulsed light 112 within the target area by solving the optical diffusion equation described in Bin Luo and Sailing He, Optics Express, Vol. 15, Issue 10, pp. 5905-5918 (2007), and storing the information in the storage unit 192. Note that as long as the light-intensity distribution can be acquired within the target area, the arithmetic operation unit 191 may acquire the light-intensity distribution by using any method.

Subsequently, the arithmetic operation unit 191 may acquire an optical-absorption-coefficient distribution within the target area as subject information in accordance with Expression (1) using the initial-sound-pressure distribution and the light-intensity distribution within the target area and stored in the storage unit 192.

Note that, in this step, the arithmetic operation unit 191 may acquire one frame of subject information from a time-series received signal obtained by performing light irradiation of one pulse of light, or may also acquire one frame of subject information from a plurality of time-series received signals obtained by performing light irradiation a plurality of times. In addition, in the case where periodic electrical stimulation is given, a piece of subject information may be acquired using received signals corresponding to one period of electrical stimulation.

In addition, for example, even in the voltage application time t1 after the delay time t2 has passed from the application start timing of the voltage, there may be the case where the S/N ratio of a received signal to be acquired is not sufficiently high at a timing at which the amount of blood is not sufficiently large. Thus, the arithmetic operation unit 191 can acquire subject information using received signals the amplitudes of which are larger than a predetermined value.

### (S500: Step for Displaying Image Based on Subject Information)

In this step, the arithmetic operation unit 191 generates image data based on the subject information acquired in S400. Here, the arithmetic operation unit 191 performs processing such as brightness-value conversion on the subject information, and generates image data for performing display on the display unit 180. Then, the arithmetic operation unit 191 transmits the generated image data of the subject information to the display unit 180, and causes the display unit 180 to display the image based on the subject information.

With the above-described subject-information acquisition method, the effect of the amount of blood for the target area on the accuracy of the subject information may be reduced.

Note that, likewise, the photoacoustic apparatus according to the present embodiment may also acquire an optical-absorption-coefficient distribution by performing the above-described steps using light of different wavelengths. Then, the arithmetic operation unit 191 may also acquire, as subject information, information on a concentration distribution of substances composing the subject 120 by using a plurality of optical-absorption-coefficient distributions corresponding to light of a plurality of wavelengths that differ from each other.

Note that in the case where, for a plurality of wavelengths, light of each wavelength is generated using a single light source, there may be the case where a certain time is needed to perform switching from one wavelength to another. Here, when one wavelength is switched to another in the state where the amount of blood is large, the number of times of light irradiation that may be performed in the state where the amount of blood is large may decrease and the accuracy of the subject information may decrease. Thus, switching from one wavelength to another can be performed in the state where the amount of blood is small. For example, when periodic electrical stimulation is given as illustrated in Part (b) of FIG. 3, the light irradiation unit 110 irradiates the subject 120 with light of a first wavelength λ1 within one period in which a periodic change as illustrated in Part (c) of FIG. 3 occurs in the amount of blood. Subsequently, a wavelength tunable mechanism within the light source 111 is driven during a period when the amount of blood is small within the one period, and the light source 111 is caused to enter a state where the light source 111 is capable of generating light of a second wavelength λ2. Subsequently, the light irradiation unit 110 irradiates the subject 120 with light of the second wavelength λ2 in the next period.

In this manner, switching of one wavelength to another can be performed in a period during which the amount of blood is small and light irradiation is determined not to be performed. As a result, irradiation of light of a plurality of wavelengths may be efficiently performed in the state where the amount of blood is large, without reducing the number of times of light irradiation that may be performed in a period during which the amount of blood is large and it is determined that light irradiation should be performed.

### Second Embodiment

Next, a subject-information acquisition method according to a second embodiment using a photoacoustic apparatus similar to that in the first embodiment will be described.

The state of muscle contraction caused by electrical stimulation changes depending on an application time or a non-application time, and thus it is considered that the way in which the amount of blood changes for a target area changes when the application time or the non-application time is changed. In addition, for example, the elasticity and configuration of blood vessels differ from subject to subject, and thus it is considered that the way in which the amount of blood changes with respect to a change in the application time or the non-application time differs from subject to subject. Thus, it is considered that, because of individual differences between subjects, the application time or the non-application time with which subject information may be acquired with high accuracy differs from subject to subject.

Thus, the present embodiment differs from the first embodiment in that a piece of desired subject information is acquired from among a plurality of pieces of subject information obtained by changing the application time t1 or the non-application time t3. In the following, configurations or steps similar to those in the first embodiment are denoted by the same reference numerals and the detailed description thereof will be omitted.

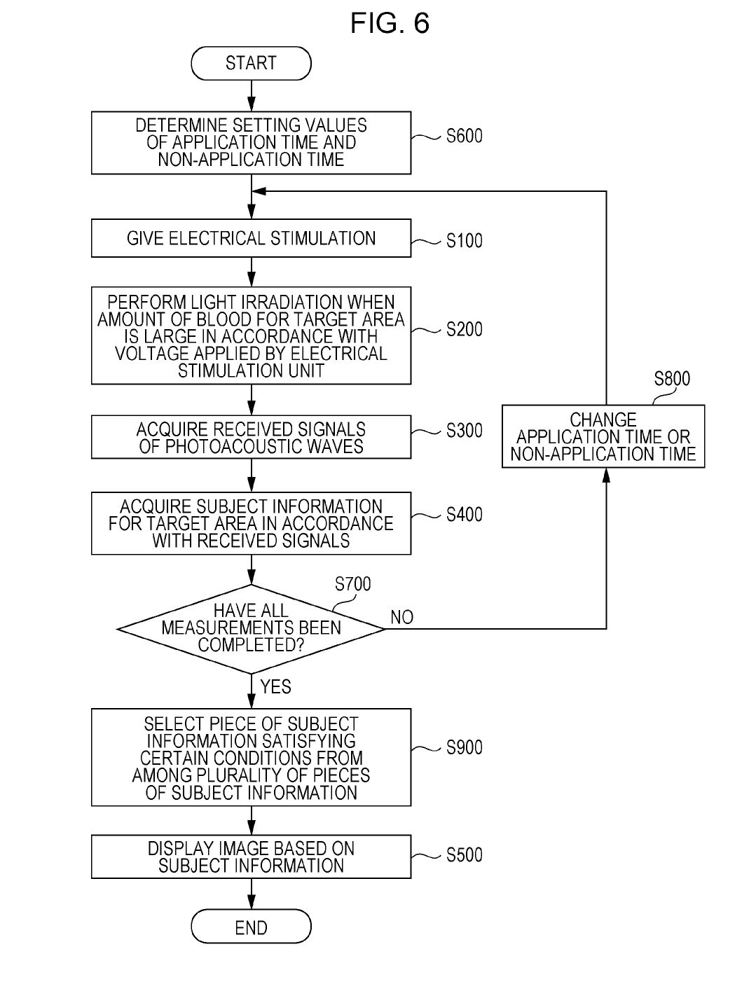

FIG. 6 illustrates a flowchart for acquiring subject information according to the second embodiment.

### (S600: Step for Determining Setting Values of Application Time and Non-Application Time)

In this step, the controller 193 determines setting values of the application time t1 and the non-application time t3 for the voltage applied by the electrical stimulation unit 150. Examples of the setting values are initial values of the application time t1 and the non-application time t3 for the voltage applied in the first measurement, and setting values of the application time t1 and the non-application time t3 changed in S800 to be described later.

The controller 193 can determine both the application time t1 and the non-application time t3 to be within the range from 10 ms to 1000 ms by taking the blood-vessel-motion reaction speed with respect to electrical stimulation into consideration. The setting values of the application time t1 and the non-application time t3 may be preset at the time of shipment, or may also be input by the user using, for example, the input unit 170.

In addition, for example, the user may input, using the input unit 170, information on a change range of the application time t1 or the non-application time t3 in S800 or the pitches of the setting values. Then, the controller 193 may determine the setting values of the application time t1 and the non-application time t3 in accordance with the information such as the input change range or the input pitches of the setting values.

In addition, the controller 193 may determine the setting values of the application time t1 and the non-application time t3 such that, in S800, the non-application time t3 is fixed and only the application time t1 is changed, or the application time t1 is fixed and only the non-application time t3 is changed.

Subsequently, the application time t1 and the non-application time t3 set in S600 are set in the electrical stimulation unit 150, and the electrical stimulation unit 150 performs electrical stimulation in the process from S100 to S400, and as a result subject information is acquired similarly to as in the first embodiment. Note that, in the present embodiment, the case will be considered where the arithmetic operation unit 191 acquires a piece of subject information from received signals obtained in response to electrical stimulation of one period. Note that received signals used to acquire a piece of subject information may be arbitrarily set.

### (S700: Step for Determining Whether or not all Measurements Have Been Completed)

In this step, the controller 193 determines whether or not all measurements for the application time t1 and the non-application time t3 determined in S600 have been completed. Then, in the case where all measurements have not been completed, the process proceeds to S800, and in the case where all measurements have been completed, the process proceeds to S900.

### (S800: Changing of Application Time or Non-Application Time)

In this step, the controller 193 sets, in the electrical stimulation unit 150, setting values of the application time t1 and the non-application time t3 that have not been set in the measurements performed so far among the setting values of the application time t1 and the non-application time t3 determined in S600.

(S900: Selection of Piece of Subject Information Satisfying Certain Conditions from Among Plurality of Pieces of Subject Information)

In this step, the controller 193 selects a piece of subject information satisfying certain conditions from among a plurality of pieces of subject information obtained by changing the application time t1 or the non-application time t3. For example, the controller 193 may select, using the resolution of subject information as a measure, a piece of subject information with which the highest resolution is realized from among a plurality of pieces of subject information. Note that in the case where a contrast or a solution has been known in advance, for example, quantitativeness may be used as a measure of an evaluation. In addition, the controller 193 can select a piece of subject information corresponding to an application time or a non-application time having a range where changes in measure are small with respect to the variation in the application time t1 or the non-application time t3.

In addition, the arithmetic operation unit 191 may cause the display unit 180 to display images based on a plurality of pieces of subject information obtained by changing the application time t1 or the non-application time t3, and the user may select a desired piece of subject information using the input unit 170. The piece of subject information selected in this manner may be treated as the piece of subject information satisfying certain conditions.

Then, the arithmetic operation unit 191 causes the display unit 180 to display the image based on the piece of subject information selected in S900 (S500).

Note that the application time t1 and the non-application time t3 set to acquire the subject information selected in S900 may be set in the electrical stimulation unit 150, and the process from S100 to S400 may be performed again. In addition, a piece of subject information can be selected by using a method different from the subject-information acquisition method performed to select a piece of subject information. For example, in the present embodiment, the piece of subject information is acquired using received signals obtained by giving electrical stimulation of one period; however, a piece of subject information may also be acquired using received signals obtained by performing electrical stimulation of a plurality of periods using the application time t1 and non-application time t3 set again. As a result, a piece of subject information having a higher S/N ratio than a piece of subject information obtained for subject-information selection may be acquired. In addition, for example, electrical stimulation of a plurality of periods is given using the application time t1 and non-application time t3 set again, and pieces of subject information may be acquired for the respective periods from received signals obtained for the plurality of periods. As a result, pieces of time-series subject information obtained when electrical stimulation is given using the application time t1 or the non-application time t3 satisfying certain conditions may be acquired.

According to the present embodiment, even in the case where there are individual differences between subjects, subject information may be acquired with high accuracy for each subject.

### Third Embodiment

Next, a subject-information acquisition method according to a third embodiment using a photoacoustic apparatus similar to that in the first or second embodiment will be described.

As described above, it is considered that because of individual differences between subjects, the application time t1 or the non-application t3 time with which subject information may be acquired with high accuracy differs from subject to subject.

Thus, the present embodiment differs from the first embodiment or the second embodiment in that the processing unit 190 changes the application time t1 or the non-application time t3 in accordance with the image data of the acquired subject information. In the following, configurations or steps similar to those in the first embodiment or the second embodiment are denoted by the same reference numerals and the detailed description thereof will be omitted.

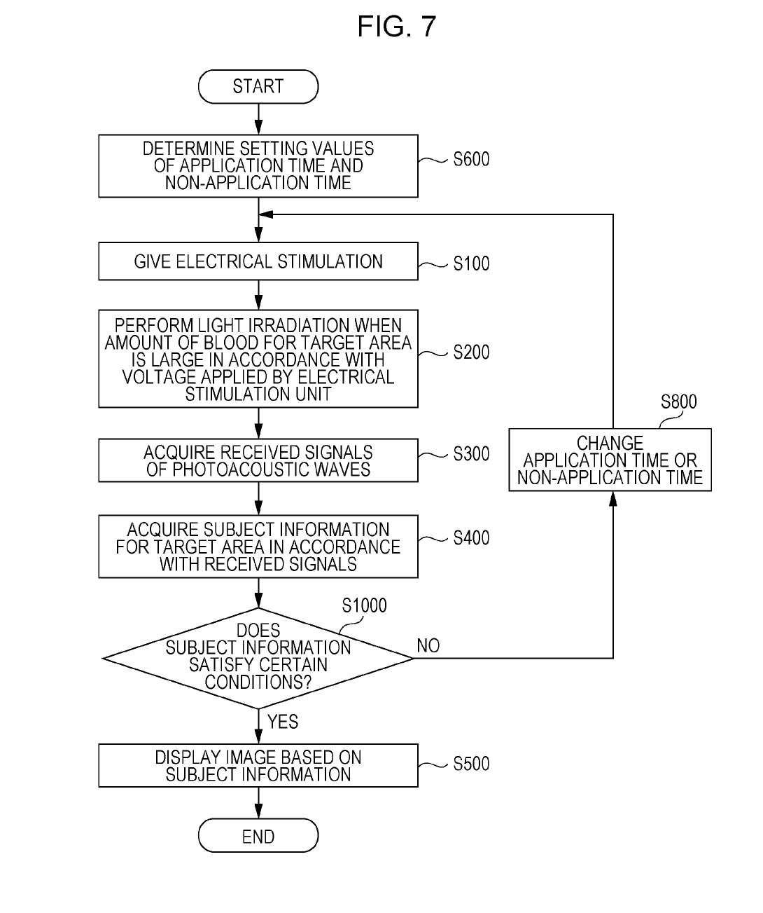

FIG. 7 illustrates a flowchart for acquiring subject information according to the third embodiment.

First, similarly to as in the second embodiment, in the process from S100 to S400, subject information is acquired by performing electrical stimulation using the application time t1 and non-application time t3 determined in S600.

### (S1000: Process for Determining Whether or not Subject Information Satisfies Certain Conditions)

In this step, first, the controller 193 determines whether or not the subject information obtained by performing electrical stimulation using the application time t1 and the non-application time t3 determined in S600 satisfies certain conditions. For example, the controller 193 calculates a measure regarding image quality such as the contrast, resolution, and quantitativeness of the subject information. In the case where it is determined that the image quality calculated by the controller 193 is in a certain range, that is, the subject information satisfies the certain conditions, the process proceeds to S500. Note that the certain conditions may be preset at the time of shipment, or may also be input by the user using the input unit 170.

Then, the arithmetic operation unit 191 generates image data in accordance with the subject information determined to satisfy the certain conditions, transmits this image data to the display unit 180, and causes the display unit 180 to display an image based on the subject information (S500).

Note that the image based on the subject information obtained in S400 is caused to be displayed on the display unit 180, and the user may input, using the input unit 170, information as to whether or not the displayed image based on the subject information satisfies the certain conditions. In this case, the process of S1000 is performed after S500.

In contrast, in the case where the controller 193 determines that the subject information does not satisfy the certain conditions, the process proceeds to S800. Then, the controller 193 sets, again, the application time t1 or the non-application time t3 of the voltage applied by the electrical stimulation unit 150 (S800).

Note that in the case where the process from S100 to S400 has been performed two times or more, the controller 193 can change the application time t1 or the non-application time t3 in accordance with the image quality of the subject information obtained in the process performed each time and information on the application time t1 or the non-application time t3 used to acquire the image quality. For example, the case is considered where the image quality has decreased when the application time t1 is changed to be longer in the second subject-information acquisition than in the first subject-information acquisition. In this case, the controller 193 may perform control such that the application time t1 is changed to be shorter in the third subject-information acquisition than in the first subject-information acquisition. In addition, also in the case where the application time t1 has been changed to be shorter in the second subject-information acquisition than in the first subject-information acquisition and in the case where the non-application time t3 is changed, the application time t1 or the non-application time t3 may be likewise changed to be longer or shorter such that improvement in image quality is expected. As a result, the number of times the process is repeatedly performed to achieve desired image quality may be reduced.

In addition, similarly to as in the second embodiment, the application time t1 and the non-application time t3 set to acquire the subject information with which the image quality is in the certain range may be set in the electrical stimulation unit 150, and the process from S100 to S400 may be performed again.

According to the present embodiment, even in the case where there are individual differences between subjects, subject information may be acquired with high accuracy for each subject.

### Other Embodiments

Embodiments of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiments and/or that includes one or more circuits (e.g., application specific integrated circuit (ASIC)) for performing the functions of one or more of the above-described embodiments, and by a method performed by the computer of the system or apparatus by, for example, reading out and executing the computer executable instructions from the storage medium to perform the functions of one or more of the above-described embodiments and/or controlling the one or more circuits to perform the functions of one or more of the above-described embodiments. The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

In the above, the details of the present invention have been described with reference to specific embodiments. However, the present invention is not limited to the above-described specific embodiments, and the embodiments may be modified without departing from the technical concepts of the present invention.

According to a photoacoustic apparatus according to the present invention, the effect of the amount of blood inside a blood vessel on the accuracy of subject information to be acquired may be reduced.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2014-242452, filed Nov. 28, 2014, which is hereby incorporated by reference herein in its entirety.

What is claimed is:

1. A photoacoustic apparatus comprising:
a light irradiation unit configured to irradiate a subject with pulsed light;
a receiving unit configured to receive a photoacoustic wave generated by irradiation of the subject with pulsed light emitted from the light irradiation unit, and output a signal;
an electrical stimulation unit configured to apply a voltage to the subject; and
a processing unit configured to set a light irradiation timing at which the light irradiation unit irradiates the subject with pulsed light, and acquire subject information for a target area in accordance with the signal, wherein
the processing unit
sets the light irradiation timing such that the subject is irradiated with pulsed light at least once in a period from an irradiation start timing determined in accordance with an application start timing of the voltage to when a first time period starting from the application start timing and ending at an application end timing of the voltage passes, and
sets the light irradiation timing such that the subject is not irradiated with pulsed light outside the period from the irradiation start timing to when the first time period passes.

2. The photoacoustic apparatus according to claim 1, wherein
the processing unit sets the light irradiation timing such that the subject is not irradiated with pulsed light outside a period from the irradiation start timing to when a time shorter than the first time period passes.

3. The photoacoustic apparatus according to claim 1, wherein
the processing unit sets the light irradiation timing such that the subject is irradiated with pulsed light the intensity of which is higher in a period from the irradiation start timing to when half the first time period passes than in a period from when the half the first time period passes from the irradiation start timing.

4. The photoacoustic apparatus according to claim 1, further comprising:
an input unit configured to be able to input at least the type of anatomical region of the target area, wherein
the processing unit
stores a relationship table regarding the type of anatomical region of the target area and a second time period starting from the application start timing and ending at the irradiation start timing,
reads out, from the relationship table, the second time period corresponding to the type of anatomical region of the target area input by the input unit, and
determines the irradiation start timing in accordance with the application start timing and the second time period read out from the relationship table.

5. The photoacoustic apparatus according to claim 1, wherein
the processing unit determines the irradiation start timing in accordance with the application start timing, information on the distance between an electrical-stimulation-target region and the target area, and information on the speed of a blood flow.

6. The photoacoustic apparatus according to claim 1, wherein
the electrical stimulation unit applies the voltage by treating a time greater than or equal to 10 ms but not greater than 1000 ms as the first time period.

7. The photoacoustic apparatus according to claim 1, wherein
the electrical stimulation unit is configured to be able to change the application start timing or the application end timing.

8. The photoacoustic apparatus according to claim 1, wherein
the light irradiation unit includes a light source configured to emit pulsed light and an optical system configured to be able to control the amount of attenuation of pulsed light emitted from the light source, and
the processing unit sets the light irradiation timing by controlling the amount of attenuation of pulsed light in the optical system in accordance with the voltage.

9. The photoacoustic apparatus according to claim 8, wherein
the processing unit sets the light irradiation timing by setting whether or not to cause the optical system to block pulsed light emitted from the light source in accordance with the voltage.

10. The photoacoustic apparatus according to claim 8, wherein
the light source emits pulsed light at a certain repetition frequency.

11. The photoacoustic apparatus according to claim 1, wherein
the light irradiation unit includes a light source configured to emit pulsed light, and
the processing unit sets the light irradiation timing by setting a light-emission timing of the light source in accordance with the voltage.

12. The photoacoustic apparatus according to claim 1, wherein
the light irradiation unit includes a light source capable of emitting pulsed light of each of a plurality of wavelengths that differ from each other, and
the light source performs switching from one wavelength to another among the plurality of wavelengths outside the period from the irradiation start timing to when the first time period passes.

13. A photoacoustic apparatus comprising:
a light irradiation unit configured to irradiate a subject with pulsed light;
an input unit configured to receive a command for driving the light irradiation unit from a user;
a receiving unit configured to receive a photoacoustic wave generated by irradiation of the subject with pulsed light emitted from the light irradiation unit, and output a signal;
an electrical stimulation unit configured to apply a voltage to the subject; and
a processing unit configured to set a light irradiation timing at which the light irradiation unit irradiates the subject with pulsed light, and acquire subject information in accordance with the signal, wherein
the processing unit
sets the light irradiation timing such that when information on the command for driving is received from the input unit, the subject is irradiated with pulsed light at least once in a period from an irradiation start timing determined in accordance with an application start timing of the voltage to when a first time period starting from the application start timing and ending at an application end timing of the voltage passes, and
sets the light irradiation timing such that the subject is not irradiated with pulsed light outside the period from the irradiation start timing to when the first time period passes or the subject is not irradiated with pulsed light when information on a command for driving is not received from the input unit.

14. A photoacoustic apparatus comprising:
a light irradiation unit configured to irradiate a subject with pulsed light;
a receiving unit configured to receive a photoacoustic wave generated by irradiation of the subject with pulsed light emitted from the light irradiation unit, and output a signal;
an electrical stimulation unit configured to apply a voltage to the subject; and
a processing unit configured to set a light irradiation timing at which the light irradiation unit irradiates the subject with pulsed light, and acquire subject information for a target area in accordance with the signal, wherein
the processing unit
sets the light irradiation timing such that the subject is irradiated with pulsed light at least once when the amount of blood for the target area is large in accordance with information on the voltage, and
sets the light irradiation timing such that the subject is not irradiated with pulsed light when the amount of blood for the target area is small in accordance with the information on the voltage.
